(19) United States
(12) Patent Application Publication
(10) Pub. No.: US 2024/0277720 A1
(43) Pub. Date: Aug. 22, 2024

(54) STABILIZED APILIMOD COMPOSITIONS AND USES THEREOF

(71) Applicant: OrphAI Therapeutics Inc., Guilford, CT (US)

(72) Inventors: Keith Fandrick, Guilford, CT (US); Paul Beckett, Guilford, CT (US); Lawrence S. Melvin, JR., Guilford, CT (US); Peter Ronald Young, Short Hills, NJ (US); Joan Edwards, Guilford, CT (US)

(21) Appl. No.: 18/410,608

(22) Filed: Jan. 11, 2024

Related U.S. Application Data
(63) Continuation of application No. 18/528,630, filed on Jan. 1, 0001.
(60) Provisional application No. 63/202,438, filed on Jun. 11, 2021.

(51) Int. Cl. A61K 31/5377; A61K 9/00; A61K 9/20

(52) U.S. Cl. CPC A61K 31/5377; A61K 9/0056; A61K 9/2018; A61K 9/2063

(57) ABSTRACT

The disclosure provides pharmaceutical compositions comprising a stabilized pharmaceutically acceptable salt of apilimod, and one or more pharmaceutically acceptable excipients, and related compositions and methods for their use in treating neurodegenerative diseases or disorders, cancer, and viral infections.

### CROSS-REFERENCE TO RELATED APPLICATIONS

This application is a continuation application of U.S. application Ser. No. 18/528,630 filed Dec. 4, 2023, now pending; which is a 35 USC § 371 National Stage application of International Application No. PCT/US2022/033107 filed Jun. 10, 2022, now expired; which claims the benefit under 35 USC § 119(e) to U.S. Application Ser. No. 63/202,438 filed Jun. 11, 2021, now expired. The disclosure of each of the prior applications is considered part of and is incorporated by reference in the disclosure of this application.

### BACKGROUND OF THE INVENTION

### Field of the Invention

The present invention relates to stabilized forms of apilimod, stabilized formulations of apilimod, and methods of using same in therapy.

### Background Information

Apilimod, also referred to as STA-5326, hereinafter “apilimod,” is recognized as a potent transcriptional inhibitor of IL-12 and IL-23. See e.g., Wada et al., Blood 109 (2007): 1156-1164. IL-12 and IL-23 are inflammatory cytokines normally produced by immune cells, such as B-cells and macrophages, in response to antigenic stimulation. Autoimmune disorders and other disorders characterized by chronic inflammation are characterized in part by inappropriate production of these cytokines. In immune cells, the selective inhibition of IL-12/IL-23 transcription by apilimod was recently shown to be mediated by apilimod's direct binding to phosphatidylinositol-3-phosphate 5-kinase (PIKfyve). See, e.g., Cai et al., Chemistry and Biol. 20 (2013):912-921, Gayle et al., Blood 129 (2017); 1768-1778. PIKfyve plays a role in Toll-like receptor signaling, which is important in innate immunity.

Based upon its activity as an immunomodulatory agent and a specific inhibitor of IL-12/IL-23, apilimod has been proposed as useful in treating autoimmune and inflammatory diseases and disorders. See e.g., U.S. Pat. Nos. 6,858,606 and 6,660,733 (describing a family of pyrimidine compounds, including apilimod, purportedly useful for treating diseases and disorders characterized by IL-12 or IL-23 overproduction, such as rheumatoid arthritis, sepsis, Crohn's disease, multiple sclerosis, psoriasis, or insulin dependent diabetes mellitus). Similarly, apilimod was suggested to be useful for treating certain cancers based upon its activity to inhibit c-Rel or IL-12/23, particularly in cancers where these cytokines were believed to play a role in promoting aberrant cell proliferation. See e.g., WO 2006/128129 and Baird et al., Frontiers in Oncology 3:1 (2013, respectively).

Each of three clinical trials of apilimod has focused on its potential efficacy in autoimmune and inflammatory diseases. The trials were conducted in patients having psoriasis, rheumatoid arthritis, and Crohn's disease. An open-label clinical study in patients with psoriasis concluded that oral administration of apilimod showed immunomodulatory activity supporting the inhibition of IL-12/IL-23 synthesis for the treatment of TH1- and TH17-mediated inflammatory diseases. Wada et al., PLosOne 7:e35069 (April 2012). But the results of controlled trials in rheumatoid arthritis and Crohn's disease did not support the notion that IL-12/IL-23 inhibition by apilimod translates into clinical improvement in either of these indications. In a randomized, double-blind, placebo-controlled Phase II clinical trial of apilimod in patients with rheumatoid arthritis, apilimod failed to alter synovial IL-12 and IL-23 expression. Krauz et al., Arthritis & Rheumatism 64:1750-1755 (2012). The authors concluded that the “results do not support the notion the IL-12/IL-23 inhibition by apilimod is able to induce robust clinical improvement in RA.” Similarly, a randomized, double-blind, placebo-controlled trial of apilimod for treatment of active Crohn's disease concluded that, although well tolerated, apilimod did not demonstrate efficacy over placebo. Sands et al., Inflamm Bowel Dis. 2010 July; 16(7):1209-18.

Disalt inhibitors of IL-12, including apilimod, are described in WO 2005112938.

### SUMMARY OF THE INVENTION

The present invention provides pharmaceutical preparations of apilimod that are stable against chemical degradation, particularly when stored under ambient conditions of 25° C. and 60% relative humidity (RH) for periods of at least 1 month, preferably for periods of from 1-3 months, from 1-6 months, or from 1-12 months. In some embodiments, the apilimod salts described herein are in a solid oral dosage form (e.g., as oral disintegrating tablets (ODT)). Further, the apilimod salts described herein (e.g., in a solid oral dosage form such as oral disintegrating tablets) are fast dissolving under acidic conditions (e.g., pH of 1-2) and have good bioavailability, while, unexpectedly, the apilimod free base ODTs dissolve slowly. Further provided herein are compositions comprising the apilimod salts, preferably monosalts, that are resistant to chemical degradation including the formation of 2-vinyl pyridine and STA-6066, and related compositions and methods for their use in therapy, including use in methods for treating neurodegenerative diseases and disorders, cancer, and viral infections. In embodiments, the neurodegenerative disease or disorder is selected from Alzheimer's disease (AD), dementia pugilistica, diffuse Lewy body disease, frontotemporal dementia (FTD), amyotrophic lateral sclerosis (ALS), mixed dementia, senile dementia of Lewy body type, Parkinson's Disease, Huntingdon's disease, and vascular dementia. In embodiments, the neurodegenerative disease or disorder is selected from frontotemporal dementia (FTD) and amyotrophic lateral sclerosis (ALS). In embodiments, the cancer is non-Hodgkin's lymphoma, follicular lymphoma, renal cancer, colorectal cancer, or melanoma. In embodiments, the viral disease is caused by a coronavirus. In embodiments, the viral infection is caused by a coronavirus. In embodiments, the coronavirus is selected from SARS-CoV-1, MERS-CoV, and SARS-CoV-2. In embodiments, the coronavirus is SARS-CoV-2. In embodiments, the viral infection is caused by an Ebola virus or a Marburg virus. In one embodiment, the virus is an Ebola virus. In one embodiment, the Ebola virus belongs to a strain selected from the group consisting of the Bundibugyo, Sudan, Tai Forest, and Zaire strains. In one embodiment, the Ebola virus is a Zaire Ebola virus.

Some aspects of the present disclosure provide pharmaceutical compositions comprising a stabilized pharmaceutically acceptable salt of apilimod, and one or more pharmaceutically acceptable excipients. In some embodiments, the apilimod is stabilized against the formation of one or more degradation products when stored under conditions of 25° C. and 60% relative humidity (RH) for at least 3 months, preferably at least 6 months. In some embodiments, the one or more degradation products is selected from one or both of 2-vinyl-pyridine and STA-6066. In some embodiments, the salt is selected from the group consisting of hydrochloride, phosphate, lactate, L-tartrate, fumarate, maleate, malonate, and glycolate. In some embodiments, the salt is a hydrochloride, malonate, or L-tartrate salt.

In some embodiments, the composition is formulated as a solid oral dosage form. In some embodiments, the solid oral dosage form is a hard or soft gelatin capsule, a tablet, an orally dissolving tablet, or a sublingual dosage form. In some embodiments, the solid oral dosage form is an orally disintegrating tablet. In some embodiments, the solid oral dosage form is fast-dissolving under acidic conditions, optionally wherein the acidic condition has a pH of 1-2. In some embodiments, the one or more pharmaceutically acceptable excipients is selected from one or more diluents, lubricants, glidants, wetting agents, disintegrants, and stabilizers. In some embodiments, the diluent is selected from one or more of mannitol, lactose, corn starch, and microcrystalline cellulose.

In some embodiments, the composition further comprises a glidant, a lubricant, or both. In some embodiments, the glidant is colloidal anhydrous silica and the lubricant is magnesium stearate.

In some embodiments, the composition further comprises a superdisintegrant. In some embodiments, the superdisintegrant is selected from the group consisting of sodium starch glycolate, croscarmellose, and crospovidone.

Other aspects of the present disclosure provide solid oral dosage forms of apilimod comprising an apilimod salt and one or more pharmaceutically acceptable excipients, wherein the apilimod salt is a hydrochloride, malonate, or L-tartrate salt of apilimod. In some embodiments, the apilimod salt is micronized.

In some embodiments, the solid oral dosage form further comprises gelatin and/or mannitol. In some embodiments, the solid oral dosage form further comprises fish gelatin and mannitol.

In some embodiments, the solid oral dosage form is obtained by lyophilizing an aqueous composition comprising 15-20% w/w of apilimod hydrochloride, 2-5% w/w of fish gelatin, 1-4% w/w of mannitol, and 72-78% w/w of water. In some embodiments, the solid oral dosage form is obtained by lyophilizing an aqueous composition comprising 18-22% w/w of apilimod malonate, 2-5% w/w of fish gelatin, 1-4% w/w of mannitol, and 70-75% w/w of water. In some embodiments, the solid oral dosage form is obtained by lyophilizing an aqueous composition comprising 21-25% w/w of apilimod tartrate, 2-5% w/w of fish gelatin, 1-4% w/w of mannitol, and 68-72% w/w of water.

In some embodiments, the solid oral dosage form is an orally disintegrating tablet. In some embodiments, the solid oral dosage form is fast-dissolving under acidic conditions. In some embodiments, the solid oral dosage form achieves at least 80% dissolution within 15 minutes under an acidic condition. In some embodiments, the acidic condition has a pH of 1-2. In some embodiments, the solid dosage form is stable for at least 3 months when stored under conditions of 25° C. and 60% relative humidity (RH).

Kits comprising the pharmaceutical composition or the solid oral dosage form of apilimod described herein are provided.

In some embodiments, the pharmaceutical composition or the solid oral dosage form of apilimod described herein are for use in treating a disease in a subject in need thereof.

In some embodiments, the pharmaceutical composition or the solid oral dosage form of apilimod described herein are for use in the manufacture of a medicament for the treatment of a disease in a subject in need thereof.

Further provided herein are methods for treating a neurodegenerative disease or disorder in a subject in need thereof, the method comprising administering to the subject the pharmaceutical composition or the solid oral dosage form of apilimod described herein.

In some embodiments, the neurodegenerative disease or disorder is a dementia. In some embodiments, the dementia is selected from AIDS dementia complex (ADC), dementia associated with Alzheimer's disease (AD), dementia pugilistica, diffuse Lewy body disease, frontotemporal dementia (FTD), mixed dementia, senile dementia of Lewy body type, and vascular dementia. In some embodiments, the neurodegenerative disease or disorder is frontotemporal dementia (FTD) or amyotrophic lateral sclerosis (ALS). In some embodiments, the subject in need of treatment is one having repeat expansions in the C9ORF72 gene. In some embodiments, the subject in need of treatment is one having a mutation in the SOD1 gene.

Also provided herein are methods for treating cancer in a subject in need thereof, the method comprising administering to the subject the pharmaceutical composition or the solid oral dosage form of apilimod described herein. In some embodiments, the cancer is selected from brain cancer, breast cancer, cervical cancer, colorectal cancer, leukemia, lung cancer, lymphoma, non-Hodgkin's lymphoma, follicular lymphoma, melanoma or other skin cancer, ovarian cancer, prostate cancer, renal cancer, pancreatic cancer, liver cancer, and testicular cancer.

Also provided herein are methods for treating a viral infection in a subject in need thereof, the method comprising administering to the subject the pharmaceutical composition or the solid oral dosage form of apilimod described herein. In some embodiments, the viral infection is caused by a coronavirus. In some embodiments, the coronavirus is selected from SARS-CoV-1, MERS-CoV, and SARS-CoV-2. In some embodiments, the viral infection is caused by an Ebola virus or a Marburg virus. In some embodiments, the subject is human.

Further aspects of the present disclosure provide methods of manufacturing a solid oral dosage form of apilimod, the method comprising mixing an apilimod salt and one or more pharmaceutically acceptable excipients, wherein the apilimod salt is a hydrochloride, malonate, or L-tartrate salt of apilimod. In some embodiments, the apilimod salt is micronized. In some embodiments, the pharmaceutically acceptable excipients comprise fish gelatin and mannitol. In some embodiments, the solid dosage form is an orally disintegrating tablet.

### BRIEF DESCRIPTION OF THE DRAWINGS

FIGS. 1A-1B: Chemical instability of apilimod dimesylate in a reference capsule formulation. (FIG. 1A) shows amount of 2-vinyl pyridine over time under various conditions: Refrigerated at 5° C. (circles), Ambient (25° C./60% RH, squares), Intermediate (30° C./60% RH, triangles), Accelerated (40° C./75% RH, diamonds); amount of 2-vinyl pyridine by determined by high pressure liquid chromatograph (HPLC); lower level for detection of 2-vinyl pyridine is 0.10%. (FIG. 1B) shows amount of another degradation product, STA-6066 over time under various conditions: Refrigerated at 5° C. (squares), Ambient (25° C./60% RH, triangles), Intermediate (30° C./60% RH, circles), Accelerated (40° C./75% RH, diamonds)

FIG. 2 shows a summary of results for kinetic solubility of the apilimod freebase and nine salts in FaSSGF (pH 1.6) at ambient temperature.

FIG. 3 shows a summary of results for the kinetic solubility of the apilimod freebase and nine salts in FaSSiF (pH 6.5) at ambient temperature.

FIG. 4 shows the dissolution profiles for micronized apilimod hydrochloride salt as oral disintegrating tablets (ODT).

FIG. 5 shows the dissolution profiles for micronized apilimod malonate salt as oral disintegrating tablets (ODT).

FIG. 6 shows the dissolution profiles for micronized apilimod L-tartrate as oral disintegrating tablets (ODT).

FIG. 7 shows the dissolution profiles for micronized apilimod free base as oral disintegrating tablets (ODT). Unexpectedly, the free base ODT was slow to dissolve.

### DETAILED DESCRIPTION OF THE INVENTION

The present inventors unexpectedly found that apilimod dimesylate, when formulated as a powder blend for an oral dosage form using common pharmaceutically acceptable excipients, is unstable at room temperature. In particular, apilimod was susceptible to chemical degradation and the formation of undesirable degradation products, including 2-vinyl pyridine and STA-6066. 2-vinyl pyridine is absorbed from the gastrointestinal tract in rodent models (mice, rats) and results in weakness, ataxia, vasodilation, respiratory distress, and convulsions. See Clayton, G. D. and F. E. Clayton (eds.). Patty's Industrial Hygiene and Toxicology: Volume 2A, 2B, 2C: Toxicology. 3rd ed. New York: John Wiley Sons, 1981-1982., p. 2735.

The present invention addresses the need for pharmaceutical preparations of apilimod that are stable against chemical degradation at ambient temperature (25° C.), especially as against the formation of 2-vinyl pyridine and STA-6066. The disclosure provides compositions comprising pharmaceutically acceptable salts of apilimod that are resistant to chemical degradation compared to a reference composition. In embodiments the reference composition is a dry powder blend of excipients and apilimod dimesylate in a gelatin capsule. The disclosure provides a number of acids that each form a crystalline solid with apilimod having a melting point above 130° C., and which are stable against polymorph formation as well as chemical degradation, particularly with respect to the formation of 2-vinyl pyridine and STA-6066, and particularly when formulated as a powder blend with common excipients.

In embodiments, the disclosure provides a pharmaceutical composition in the form of dry powder blend comprising a pharmaceutically acceptable monosalt of apilimod and one or more pharmaceutically acceptable excipients, wherein the composition comprises less than 0.2% w/w of an apilimod degradation product. In embodiments, the composition comprises less than 0.05%, less than 0.1%, or less than 0.2% w/w of an apilimod degradation product following exposure to ambient conditions, i.e., a controlled temperature and relative humidity (RH) of 25° C./60% RH for a period of from 1-3 months or from 1-6 months. In embodiments, the apilimod degradation product is selected from one or both of 2-vinyl pyridine and STA-6066.

The structure of apilimod free base is shown in Formula I:

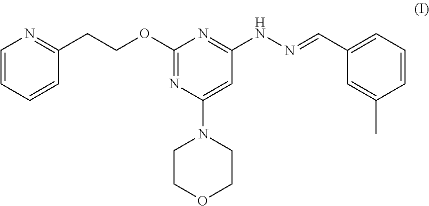 [SMILES: Cc1cccc(/C=N/Nc2cc(N3CCOCC3)nc(OCCc3ccccn3)n2)c1]

The chemical name of apilimod is 2-[2-Pyridin-2-yl)-ethoxy]-4-N′-(3-methyl-benzilidene)-hydrazino]-6-(morpholin-4-yl)-pyrimidine (IUPAC name: (E)-4-(6-(2-(3-methylbenzylidene)hydrazinyl)-2-(2-(pyridin-2-yl)ethoxy)pyrimidin-4-yl)morpholine), and the CAS number is 541550-19-0. Apilimod can be prepared, for example, according to the methods described in U.S. Pat. Nos. 7,923,557, and 7,863,270, and WO 2006/128129.

The dimesylate salt form of apilimod was initially selected for development due its high solubility in water (831 mg/mL) and physical stability. See e.g., WO 2005112938. However, in contrast to the stability of the apilimod dimesylate salt form itself, the present inventors found that when blended with typical solid excipients for use as a dry powder in a capsule dosage form, the apilimod degraded primarily into 2-vinyl pyridine and STA-6066.

Accordingly, the present invention provides monosalts, i.e., salts having a 1:1 stoichiometry of acid to apilimod. The monosalts described here form a less acidic salt compared to the dimesylate form and are relatively more stable when formulated as a dry powder with common excipients at under ambient conditions. Monosalts of apilimod and suitable acids were prepared by heating a solution of apilimod in an appropriate solvent to 50° C. and adding 1 equivalent of the acid. The solution was cooled to ambient temperature and stirred overnight. Crystallinity of the salt was confirmed using X-ray powder diffraction analysis. The salts were subsequently dried, either by exposure to air or vacuum dried in a 50° C. vacuum oven with a nitrogen bleed, or a combination.

In some embodiments, the apilimod salt described herein is selected from the group consisting of hydrochloride, phosphate, lactate, L-tartrate, fumarate, maleate, malonate, and glycolate salt. In some embodiments, the apilimod salt described herein is a hydrochloride, malonate, or L-tartrate salt.

In embodiments, the apilimod is micronized. Micronization of drug particles can be achieved by mechanical means, such as milling, for example by fluid energy or jet-milling, pin-milling, wet-polishing, ball and or pebble mill, edge runner mill, rotary cutter mill, end runner mill, roller mill, hammer mill, mortor and pestle, colloid mill, etc. Other technologies to generate micronized drug particles include mechanical communication, spray drying, and supercritical fluid (SFC). In addition, in-situ techniques to directly generate micron or sub-micron sized crystals can be employed.

In embodiments, the apilimod is subjected to nanonization. Suitable methods for nanonization include ultraosonic precipitation, bead milling, high pressure homogenization, media milling, and dry co-grinding.

### Pharmaceutical Compositions and Formulations

The disclosure provides stabilized salt forms of apilimod and pharmaceutical compositions comprising same. In this context, “stabilized” refers to stabilization against chemical degradation of apilimod and the formation of degradation products such as 2-vinyl-pyridine and STA-6066.

In embodiments, the disclosure provides a pharmaceutical composition in the form of a hard or soft gelatin capsule, a tablet, an orally disintegrating tablet, or a sublingual dosage form comprising apilimod and one or more excipients. In embodiments, the disclosure provides a pharmaceutical composition in the form of a hard or soft gelatin capsule or tablet comprising a dry powder blend of apilimod and one or more excipients. In accordance with this embodiment, the one or more excipients may be selected from one or more diluents, lubricants, glidants, wetting agents, disintegrants, and stabilizers. The terms “diluent”, “filler”, and “bulking agent” are used interchangeably in this context. In accordance with this embodiment, the amount of apilimod in the powder blend is from 10-60 wt %, with the remainder being filled by the one or more excipients. In embodiments, the one or more excipients includes a diluent or a combination of diluents. Suitable diluents include lactose, corn starch, and microcrystalline cellulose. In embodiments, the one or more excipients further includes, in addition to diluent, a glidant, a lubricant, or both a glidant and a lubricant. In general, glidants are materials that reduce interparticulate friction, such as colloidal anhydrous silica; and lubricants are materials reduce adhesion of the powder to metal, such as magnesium stearate. In embodiments, the one or more excipients may further comprise a wetting agent, such as sodium lauryl sulphate, and a disintegrant, preferably a superdisintegrant such as sodium starch glycolate, croscarmellose, or crospovidone.

Examples of suitable diluents for tablets or capsules include calcium carbonate, calcium lactate, calcium phosphate, calcium silicate, calcium sulfate, cellaburate, cellulose acetate, cellulose microcrystalline, cellulose powdered, cellulose silicified microcrystalline, corn starch, corn syrup solids, dextrates, dextrin, dextrose, erythritol, ethylcellulose, glyceryl palmitostearate, hydroxypropyl cellulose, inulin, kaolin, lactitol, lactose, lactose monohydrate and povidine coprocessed, lactose monohydrate and powdered cellulose, magnesium carbonate, magnesium oxide, maltitol, maltodextrin, maltose, maltodextrin, maltose, mannitol, medium chain triglycerides, polydextrose, polyethylene glycol, propylparaben sodium, simethicone, sodium bicarbonate, sodium carbonate, sodium chloride, sorbitol, starch, sucrose, sugar, sunflower oil, talc, trehalose, xylitol.

Examples of suitable disintegrants for tablets or capsules include agar, alginic acid, asparagine, calcium alginate, carboxymethylcellulose calcium, carboxymethylcellulose sodium, cellulose, ceratonia, chitosan, colloidal silicon dioxide, corn starch and pregelatinized starch, croscarmellose sodium, crospovidone, glycine, guar gum, hydroxypropyl cellulose, hydroxypropyl starch, lactose monohydrate and corn starch, magnesium aluminum, maltose, methylcellulose, polacrilin potassium, povidone, sodium alginate, sodium starch glycolate, starch.

Examples of suitable binders for tablets or capsules include acacia, agar, alginic acid, ammonium alginate, attapulgite, calcium carbonate, calcium lactate, calcium polycarbophil, carboxymethylcellulose calcium, carboxymethyl cellulose sodium, cellulose acetate phthalate, ceratonia, chitosan, colophony, copovidone, corn syrup solids, dextrates, dextrin, dextrose, dextrose anhydrous, ethylcellulose, ethylene glycol and vinyl alcohol grafted copolymer, gelatin, glucose, glycerin behenate, guar gum, hydroxyethyl cellulose, hydroxypropyl cellulose, hydroxyethylmethyl cellulose, hypromellose, isomalt, lactose monohydrate, magnesium aluminum silicate, maltitol, maltodextrin, maltose, methylcellulose, polycarbophil, polydextrose, polyethylene oxide, polymethacrylates, povidone, propylparaben sodium, sodium alginate, starch, sucrose, sugar, vegetable oil, vitamin E polyethylene glycol succinate, zein.

Examples of suitable lubricants for tablets or capsules include calcium stearate, castor oil, glyceryl behenate, glyceryl monostearate, glyceryl palmitostearate, leucine, magnesium stearate, mineral oil, myristic acid, palm oil, palmitic acid, poloxamer, polyethylene glycol, potassium glycol, potassium benzoate, sodium benzoate, sodium lauryl sulfate, sodium stearate, sodium stearate fumarate, stearic acid, sucrose stearate, talc, vegetable oil, zinc stearate.

Examples of suitable glidants for tablets or capsules include cellulose, colloidal silicon dioxide, hydrophobic colloidal silica, magnesium oxide, magnesium silicate, magnesium trisilicate, sodium stearate, and talc.

In some embodiments, the apilimod salt described herein is in a solid oral dosage form. In some embodiments, the solid oral dosage form is an orally disintegrating tablet (ODT). In some embodiments, the apilimod salt described herein is a hydrochloride, malonate, or L-tartrate salt in a solid oral dosage form, e.g., as orally disintegrating tablets. In some embodiments, the solid oral dosage form (e.g., as orally disintegrating tablets) are fast-dissolving under acidic conditions.

In some embodiments, the solid oral dosage form (e.g., as orally disintegrating tablets) of the apilimod salts (e.g., an apilimod hydrochloride, malonate, or L-tartrate salt) described herein further comprises one or more pharmaceutically acceptable excipients. In some embodiments, the one or more pharmaceutically acceptable excipients comprise gelatin (e.g., fish gelatin) and/or mannitol. In some embodiments, the one or more pharmaceutically acceptable excipients comprise fish gelatin and mannitol.

In some embodiments, the solid oral dosage form (e.g., as orally disintegrating tablets) of the apilimod salts described herein comprises an apilimod salt (e.g., an apilimod hydrochloride, malonate, or L-tartrate salt), fish gelatin, and mannitol. In some embodiments, the solid oral dosage form (e.g., as orally disintegrating tablets) of the apilimod salts described herein is obtained by lyophilizing an aqueous composition comprising an apilimod salt (e.g., an apilimod hydrochloride, malonate, or L-tartrate salt), fish gelatin, mannitol, and water. In some embodiments, the solid oral dosage form (e.g., as orally disintegrating tablets) of the apilimod salts described herein is obtained by lyophilizing an aqueous composition comprising 15-25% w/w (e.g., 15-25% w/w, 15-22.5 w/w, 15-20% w/w, 15-17.5% w/w, 17.5-25% w/w, 17.5-22.5% w/w, 17.5-20% w/w, 20-25% w/w, 20-22.5% w/w, or 22.5-25% w/w) of an apilimod salt (e.g., an apilimod hydrochloride, malonate, or L-tartrate salt), 2-5% w/w (e.g., 2-5, 2-4, 2-3, 3-5, 3-4, 4-5, 2.5-4.5, 2-4, 1.5-3.5, or 3.5-4% w/w) of fish gelatin, 1-4% w/w (e.g., 1-4, 1-3, 1-2, 2-4, 2-3, 3-4, 1.5-3.5, 2-3, or 2.5-3% w/w) of mannitol, and 70-80% w/w (e.g., 70-80% w/w, 70-75% w/w, or 75-80% w/w) of water.

In some embodiments, the solid oral dosage form (e.g., as orally disintegrating tablets) of the apilimod salts described herein is obtained by lyophilizing an aqueous composition comprising 15-20% w/w (e.g., about 15, 15.5, 16, 16.5, 17, 17.5, 17.86, 18, 18.5, 19, 19.5, or 20% w/w) of apilimod hydrochloride, 2-5% w/w (e.g., about 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0% w/w) of fish gelatin, 1-4% w/w (e.g., about 2.1, 2.2, 2.3, 2.4, 2.5, 2.6, 2.7, 2.8, 2.9, or 3% w/w) of mannitol, and 72-78% w/w (e.g., about 72, 72.5, 73, 73.5, 74, 74.5, 75, 75.5, 75.64, 76, 76.5, 77, 77.5, or 78% w/w) of water. In some embodiments, the solid oral dosage form is obtained by lyophilizing an aqueous composition comprising 15-20% w/w of apilimod hydrochloride, 3.5-3.8% w/w of fish gelatin, 2.5-3% w/w of mannitol, and 72-78% w/w of water.

In some embodiments, the solid oral dosage form (e.g., as orally disintegrating tablets) of the apilimod salts described herein is obtained by lyophilizing an aqueous composition comprising 18-22% w/w (e.g., about 18, 18.5, 19, 19.5, 20, 20.5, 20.99, 21, 21.5, or 22% w/w) of apilimod malonate, 2-5% w/w (e.g., about 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0% w/w) of fish gelatin, 1-4% w/w (e.g., about 2.1, 2.2, 2.3, 2.4, 2.5, 2.6, 2.7, 2.8, 2.9, or 3% w/w) of mannitol, and 70-75% w/w (e.g., about 70, 70.5, 71, 71.5, 72, 72.5, 72.51, 73, 73.5, 74, 74.5, or 75% w/w) of water. In some embodiments, the solid oral dosage form is obtained by lyophilizing an aqueous composition comprising 18-22% w/w of apilimod malonate, 3.5-3.8% w/w of fish gelatin, 2.5-3% w/w of mannitol, and 70-75% w/w of water.

In some embodiments, the solid oral dosage form (e.g., as orally disintegrating tablets) of the apilimod salts described herein is obtained by lyophilizing an aqueous composition comprising 21-25% w/w (e.g., about 21, 21.5, 22.5 23, 23.05, 23.5, 24, 24.5, or 25,% w/w) of apilimod tartrate, 2-5% w/w (e.g., about 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0% w/w) of fish gelatin, 1-4% w/w (e.g., about 2.1, 2.2, 2.3, 2.4, 2.5, 2.6, 2.7, 2.8, 2.9, or 3% w/w) of mannitol, and 68-72% w/w (e.g., about 68, 68.5, 69, 69.5, 70, 70.5, 71, 71.5, or 72% w/w) of water. In some embodiments, the solid oral dosage form is obtained by lyophilizing an aqueous composition comprising 21-25% w/w of apilimod tartrate, 3.5-3.8% w/w of fish gelatin, 2.5-3% w/w of mannitol, and 68-72% w/w of water.

In some embodiments, any one of the solid oral dosage forms (e.g., as orally disintegrating tablets) of the apilimod salts (e.g., an apilimod hydrochloride, malonate, or L-tartrate salt) are lyophilized and does not contain water (e.g., lyophilization removes water from the aqueous solution). In some embodiments, in any one of the solid oral dosage form (e.g., as orally disintegrating tablets) of the apilimod salts (e.g., an apilimod hydrochloride, malonate, or L-tartrate salt), the apilimod salt is micronized.

In some embodiments, any one of the solid oral dosage forms (e.g., as orally disintegrating tablets) of the apilimod salts (e.g., an apilimod hydrochloride, malonate, or L-tartrate salt) described herein may be produced as described in, e.g., U.S. Pat. Nos. 7,972,621, 9,192,580, U.S. Ser. No. 10/548,839, and U.S. Ser. No. 10/828,261, the entire contents of each of which are incorporated herein by reference.

A “pharmaceutical composition” is a formulation containing an active pharmaceutical ingredient, or “API”, such as apilimod, and one or more pharmaceutically acceptable excipients in a form suitable for administration to a subject for therapy. The term “pharmaceutically acceptable excipient” refers to an excipient useful in preparing a pharmaceutical composition that is generally safe, non-toxic and neither biologically nor otherwise undesirable, and includes excipients that are acceptable for veterinary use as well as human pharmaceutical use. Examples of pharmaceutically acceptable excipients include, without limitation, sterile liquids, water, buffered saline, ethanol, polyol (for example, glycerol, propylene glycol, liquid polyethylene glycol and the like), oils, detergents, suspending agents, carbohydrates (e.g., glucose, lactose, sucrose or dextran), antioxidants (e.g., ascorbic acid or glutathione), chelating agents, low molecular weight proteins, or suitable mixtures thereof.

Pharmaceutical compositions can take various different forms, for example liquids, aerosols, solutions, inhalants, mists, sprays; or solids, powders, ointments, pastes, creams, lotions, gels, patches, etc. The particular form is generally adapted for administration by a desired route, such as pulmonary, inhalation, intranasal, oral, buccal, sublingual, parenteral, subcutaneous, intravenous, intramuscular, intraperitoneal, intrapleural, intrathecal, transdermal, transmucosal, rectal, etc. For example, a pharmaceutical composition may be in the form of an aqueous solution or powder for aerosol administration by inhalation or insufflation (either through the mouth or the nose), or in the form of a tablet or capsule for oral administration; or in the form of a sterile aqueous solution or dispersion for administration by either direct injection or by addition to sterile infusion fluids for intravenous infusion; or in the form of a lotion, cream, foam, patch, suspension, solution, or suppository for transdermal or transmucosal administration.

In embodiments, the pharmaceutical composition is an oral dosage form including, but not limited to, capsules, tablets, buccal forms, troches, lozenges, and oral liquids in the form of emulsions, aqueous suspensions, dispersions or solutions. Capsules may contain mixtures of the API with inert fillers and/or diluents such as the pharmaceutically acceptable starches (e.g., corn, potato or tapioca starch), sugars, artificial sweetening agents, powdered celluloses, such as crystalline and microcrystalline celluloses, flours, gelatins, gums, etc. Lubricating agents, such as magnesium stearate, can also be added. When aqueous suspensions and/or emulsions are administered orally, the API may be suspended or dissolved in an oily phase is combined with emulsifying and/or suspending agents. If desired, certain sweetening and/or flavoring and/or coloring agents may be added.

Additional pharmaceutically acceptable excipients include diluents, binding agents, lubricants, disintegrants, surface modifying agents (including surfactants), suspending or stabilizing agents, include magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Preferred surface modifying agents include nonionic and anionic surface modifying agents. Representative examples of surface modifying agents include, but are not limited to, poloxamer 188, benzalkonium chloride, calcium stearate, cetostearyl alcohol, cetomacrogol emulsifying wax, sorbitan esters, colloidal silicon dioxide, phosphates, sodium dodecylsulfate, magnesium aluminum silicate, and triethanolamine.

A pharmaceutical composition can be provided in bulk or in dosage unit form. It is especially advantageous to formulate pharmaceutical compositions in unit dosage form for ease of administration and uniformity of dosage. The term “unit dosage form” refers to physically discrete units suitable as unitary dosages for the subject to be treated; each unit containing a predetermined quantity of API calculated to produce the desired therapeutic effect in association with the required pharmaceutical carrier. A unit dosage form can be, for example, an ampoule, a vial, a suppository, a dragee, a tablet, a capsule, an IV bag, or a single pump on an aerosol inhaler. In embodiments of the pharmaceutical compositions described here, the unit dosage form is a capsule.

In the context of the present disclosure, a unit dosage form will typically contain the API, such as apilimod, in a range of from 1-1,000 mg, preferably from 25-500 mg. For example, the unit dosage form may contain apilimod in an amount of 25 mg, 50 mg, 100 mg, 125 mg, 150 mg, 200 mg, 250 mg, 300 mg, 350 mg, 400 mg, 450 mg, or 500 mg. In embodiments, the pharmaceutical composition comprises apilimod in a unit dose of 100 mg, 125 mg, or 200 mg.

In some embodiments, the solid oral dosage form (e.g., as orally disintegrating tablets) is stable for at least 1 months (e.g., at least 1, 2, 3, 4, 5, 6, 7, 8, 9, 10, 11, 12 months or longer) when stored under conditions of about 25° C. and about 60% relative humidity (RH). In some embodiments, the solid oral dosage form (e.g., as orally disintegrating tablets) is stable for at least 3 months when stored under conditions of about 25° C. and about 60% relative humidity (RH). In some embodiments, the solid oral dosage form (e.g., as orally disintegrating tablets) is stable for at least 6 months (e.g., at least 1, 2, 3, 4, 5, 6, 7, 8, 9, 10, 11, 12 months or longer) when stored under conditions of about 25° C. and about 60% relative humidity (RH). In some embodiments, the stability is measured and/or analyzed by HPLC.

In some embodiments, the solid oral dosage form (e.g., as orally disintegrating tablets) exhibit high bioavailability. In some embodiments, the solid oral dosage form (e.g., as orally disintegrating tablets) achieves at least 60% (e.g., at least 60%, at least 65%, at least 70%, at least 75%, at least 80%, at least 85%, at least 90%, at least 95%, or higher) dissolution within 20 (e.g., within 20, 15, 10, or 5) minutes under an acidic condition. In some embodiments, the solid oral dosage form (e.g., as orally disintegrating tablets) achieves at least 80% dissolution within 15 minutes under an acidic condition. In some embodiments, wherein the acidic condition has a pH of 1-2 (e.g., 1, 1.1, 1.2, 1.3, 1.4, 1.5, 1.6, 1.7, 1.8, 1.9, or 2).

The disclosure also provides pharmaceutical compositions and/or solid oral dosage forms of apilimod as described herein (e.g., as orally disintegrating tablets), for use in treating a disease in a subject in need thereof.

The disclosure also provides pharmaceutical compositions and/or solid oral dosage forms of apilimod as described herein (e.g., as orally disintegrating tablets), for use in the manufacture of a medicament for the treatment of a disease in a subject in need thereof.

The disclosure also provides packaging and kits comprising pharmaceutical compositions for use in the methods of the present invention. The kit can comprise one or more containers selected from the group consisting of a bottle, a vial, an ampoule, a blister pack, and a syringe. The kit can further include one or more of instructions for use in treating and/or preventing a disease, condition or disorder of the present invention, one or more syringes, one or more applicators, or a sterile solution suitable for reconstituting a pharmaceutical composition of the present invention.

All percentages and ratios used herein, unless otherwise indicated, are by weight. Other features and advantages of the present invention are apparent from the different examples. The provided examples illustrate different components and methodology useful in practicing the present invention. The examples do not limit the claimed invention. Based on the present disclosure the skilled artisan can identify and employ other components and methodology useful for practicing the present invention.

### Methods of Treatment

The present disclosure provides methods for treating a neurodegenerative disease or disorder, or a cancer, in a subject in need thereof comprising administering a pharmaceutical composition comprising a stabilized salt form of apilimod, as described herein, to a subject in need of such treatment. The present disclosure further provides the use of such stabilized salt forms of apilimod, for the preparation of a medicament useful for the treatment of a neurodegenerative disease or disorder, or a cancer.

Neurodegenerative diseases and disorders that may be treated according to the methods described here include, for example, Alzheimer's disease (AD), amyotrophic lateral sclerosis (ALS), diffuse Lewy body disease, Lewy body dementia, motor neuron diseases, multiple sclerosis (MS), Parkinson's disease (PD), Friedreich's ataxia, prion disease, spinocerebellar ataxia (SCA), and spinal muscular atrophy (SMA). Other, less common neurodegenerative diseases and disorders that may be treated include, for example, Creutzfeldt-Jakob disease (CJD), progressive supranuclear palsy (PSP, Steele-Richardson-Olszewski syndrome), senile chorea, Huntington's Chorea, spinal ataxia including spinocerebellar ataxia (SCA), Friedreich's ataxia, Subacute sclerosing panencephalitis, frontotemporal dementia (also referred to as FTD, or frontotemporal lobar degeneration), and Hallerrorden-Spatz disease (Pantothenate kinase-associated neurodegeneration, PKAN).

In an embodiment, the neurodegenerative disease or disorder is ALS. In an embodiment for the treatment of ALS or frontotemporal dementia, the patient in need of treatment of is one having repeat expansions in the C9ORF72 gene. The GGGGCC repeat expansion in the C9ORF72 gene is the most common genetic cause of amyotrophic lateral sclerosis (ALS), accounting for about 10% of all ALS cases worldwide and 10% familial frontotemporal dementia (FTD). The repeat expansion generates neurotoxic species including dipeptide repeat proteins (DPRs), nuclear RNA foci, and RNA/DNA G-quadruplexes. The repeat expansion also suppresses the production of C9ORF72 protein, a protein that normally regulates vesicle trafficking and lysosomal biogenesis. In human induced motor neurons, the repeat expansion in C9ORF72 triggered neurodegeneration through two mechanisms: accumulation of glutamate receptors and impaired clearance of neurotoxic dipeptide repeat proteins. In an embodiment for the treatment of ALS, the patient in need of treatment of is one having a mutation in SOD1, another common genetic cause. In an embodiment for the treatment of ALS or frontotemporal dementia, the patient in need of treatment of is one having an accumulation of TDP-43 aggregates, a product of the TARDBP gene, found in many sporadic and familial ALS.

Various forms of dementia may also be considered neurodegenerative diseases. In general, the term ‘dementia’ describes a group of symptoms affecting memory, thinking, language, speech, and social abilities severely enough to interfere with daily functioning. Accordingly, the disclosure also provides methods of treating dementia, including AIDS dementia complex (ADC), dementia associated with Alzheimer's disease (AD), dementia pugilistica, diffuse Lewy body disease, frontotemporal dementia, mixed dementia, senile dementia of Lewy body type, and vascular dementia. In an embodiment, the dementia is frontotemporal dementia. In an embodiment for the treatment of frontotemporal dementia, the patient in need of treatment of is one having repeat expansions in the C9ORF72 gene.

Neuromuscular disorders that may be treated according to the methods described here include, for example, infantile spinal muscular atrophy (SMA 1, Werdnig-Hoffmann disease), and juvenile spinal muscular atrophy (SMA3, Kugelberg-Welander disease).

In embodiments for the treatment of Alzheimer's disease, the methods may comprise combination therapy with apilimod as part of a therapeutic regimen including administration of a cholinesterase inhibitor (e.g., Aricept™, Exelon™, Razadyne™), or a glutamatergic agent selected from memantine (Namenda™), riluzole, and trigriluzole.

In embodiments for the treatment of amyotrophic lateral sclerosis (ALS), the methods may comprise combination therapy with apilimod as part of a therapeutic regimen including administration of an antioxidant, such as edaravone (Radicava™, Radicut™). In an embodiment for the treatment of ALS, the patient in need of treatment of is one having repeat expansions in the C9ORF72 gene. In an embodiment for the treatment of ALS, the patient in need of treatment of is one having a mutation in the SOD1 gene. In an embodiment for the treatment of ALS, the patient in need of treatment of is one demonstrating aggregation of TDP-43.

In embodiments, the disclosure provides a method of treating Parkinson's disease, Parkinsonism syndrome, or multiple sclerosis in a subject in need thereof, the method comprising administering to the subject a pharmaceutical composition comprising a stabilized salt form of apilimod, as described herein.

The disclosure also provides methods of treating cancer, the methods comprising administering to the subject a pharmaceutical composition comprising a stabilized salt form of apilimod, as described herein. In embodiments, the cancer is selected from brain cancer, glioma, sarcoma, breast cancer, lung cancer, non-small-cell lung cancer, mesothelioma, appendiceal cancer, genitourinary cancers, renal cell carcinoma, prostate cancer, bladder cancer, testicular cancer, penile cancer, cervical cancer, ovarian cancer, von Hippel Lindau disease, head and neck cancer, gastrointestinal cancer, hepatocellular carcinoma, gallbladder cancer, esophageal cancer, gastric cancer, colorectal cancer, pancreatic cancer, liver cancer, melanoma, neuroendocrine tumors, thyroid tumor, pituitary tumor, adrenal tumor, hematological malignancy, or leukemia.

In embodiments the cancer is a lymphoma. In embodiments, the lymphoma is a B cell lymphoma. In embodiments, the B cell lymphoma is selected from the group consisting of a Hodgkin's B cell lymphoma and a non-Hodgkin's B cell lymphoma. In embodiments, the B cell lymphoma is a non-Hodgkin's B cell lymphoma selected from the group consisting of DLBCL, follicular lymphoma, marginal zone lymphoma (MZL) or mucosa associated lymphatic tissue lymphoma (MALT), small cell lymphocytic lymphoma (overlaps with chronic lymphocytic leukemia) and mantle cell lymphoma. In embodiments, the B cell lymphoma is a non-Hodgkin's B cell lymphoma selected from the group consisting of Burkitt lymphoma, Burkitt lymphoma, Primary mediastinal (thymic) large B-cell lymphoma, Lymphoplasmacytic lymphoma, which may manifest as Waldenström macroglobulinemia, Nodal marginal zone B cell lymphoma (NMZL), Splenic marginal zone lymphoma (SMZL), Intravascular large B-cell lymphoma, Primary effusion lymphoma, Lymphomatoid granulomatosis, T cell/histiocyte-rich large B-cell lymphoma, Primary central nervous system lymphoma, Primary cutaneous diffuse large B-cell lymphoma, leg type (Primary cutaneous DLBCL, leg type), EBV positive diffuse large B-cell lymphoma of the elderly, Diffuse large B-cell lymphoma associated with inflammation, Intravascular large B-cell lymphoma, ALK-positive large B-cell lymphoma, and Plasmablastic lymphoma. In an embodiment, the cancer is a non-Hodgkins lymphoma or a follicular lymphoma.

The disclosure also provides methods of treating a viral infection. In embodiments, the viral infection is caused by a coronavirus. In embodiments, the coronavirus is selected from SARS-CoV-1, MERS-CoV, and SARS-CoV-2. In embodiments, the coronavirus is SARS-CoV-2. In embodiments, the viral infection is caused by an Ebola virus or a Marburg virus. In one embodiment, the virus is an Ebola virus. In one embodiment, the Ebola virus belongs to a strain selected from the group consisting of the Bundibugyo, Sudan, Tai Forest, and Zaire strains. In one embodiment, the Ebola virus is a Zaire Ebola virus.

A “subject in need thereof” refers to a subject in need of treatment for a neurodegenerative disease or disorder, or a cancer. In embodiments, the subject in need is one that is “non-responsive” or “refractory” to a standard therapy for the neurodegenerative disease or disorder, or the cancer. In this context, the terms “non-responsive” and “refractory” refer to the subject's response to therapy as not clinically adequate to relieve one or more symptoms associated with the neurodegenerative disease or disorder, or the cancer. In embodiments, the patient in need of treatment of is one having repeat expansions in the C9ORF72 gene, for example, in embodiments relating to a neurodegenerative disease or disorder, especially amyotrophic lateral sclerosis (ALS) or frontotemporal dementia (FTD).

A “subject” refers generally to a mammal. The mammal can be e.g., a human, primate, mouse, rat, dog, cat, cow, horse, goat, camel, sheep or a pig. Preferably, the subject is a human. The terms “subject” and “patient” are used interchangeably herein.

The terms, “treatment”, “treating” or “treat” describes the management and care of a subject having a neurodegenerative disease or disorder, or a cancer, as described here and includes the administration of a therapeutic agent, or combination thereof as described here, to slow the progression of the disease or disorder and/or to alleviate one or more symptoms of the neurodegenerative disease or disorder, or the cancer. In this context, treating includes administering an amount of the therapeutic agent, or combination of agents, effective to alleviate one or more symptoms of the neurodegenerative disease or disorder, or the cancer. The term “alleviate” refers to a process by which the severity of a symptom is reduced or decreased, but it may not necessarily be eliminated, although it may be eliminated for a period of time, or temporarily. While elimination of the symptom is preferred, it is not required. The terms, “prevention”, “preventing” or “prevent” refer to reducing or eliminating the onset of a symptom, especially in the context of preventing the progression of the disease or disorder, or the cancer, where progression is defined by the onset one or more symptoms.

The term “therapeutically effective amount” refers to an amount sufficient to treat, ameliorate a symptom of, reduce the severity of, or reduce the duration of the neurodegenerative disease or disorder, or the cancer, or to enhance or improve the therapeutic effect of another therapy. The precise effective amount for a subject will depend upon the subject's body weight, size, and health; the nature and extent of the condition; and the therapeutic or combination of therapeutics selected for administration.

In embodiments a therapeutically effective amount of apilimod for use in the treatment of a neurodegenerative disease or disorder, a cancer, or a viral infection in an adult human is from 100 to 400 mg/day, preferably from about 150 to 250 mg/day. In embodiments, the therapeutically effective amount of apilimod for use in the treatment of a neurodegenerative disease or disorder, a cancer, or a viral infection is 150, 200, 250, or 300 mg/day. In embodiments of the methods described here, the pharmaceutical composition may comprise 75, 100, or 125 mg apilimod for administration twice daily in an adult human subject.

In accordance with the therapeutic methods described here, the apilimod may be administered as monotherapy, in which the apilimod is the only API administered, in the treatment of a neurodegenerative disease or disorder, or a cancer. The methods described here may also comprise combination therapy with apilimod and at least one additional API. The terms, “combination therapy” or “co-therapy” include the administration of a compound described herein, e.g., apilimod, with at least one additional API, as part of a specific treatment regimen intended to provide a beneficial effect from the co-action of the two APIs. The beneficial effect may result in the slowing of the progression of the neurodegenerative disease or disorder, or the cancer, and/or the alleviation of one or more symptoms of the neurodegenerative disease or disorder, or the cancer. The beneficial effect of the combination includes, but is not limited to, pharmacokinetic or pharmacodynamic co-action resulting from the combination. The beneficial effect of the combination may also relate to the mitigation of a toxicity, side effect, or adverse event associated with another agent in the combination. “Combination therapy” is not intended to encompass the administration of two or more of these therapeutic compounds as part of separate monotherapy regimens that incidentally and arbitrarily result in the combinations of the present disclosure.

In the context of combination therapy, administration of the API apilimod may be simultaneous with or sequential to the administration of the additional API, or administration may be prior to, concomitantly with, or subsequent to the administration of the additional API. The different APIs of a combination therapy can be formulated for co-administration in a single dosage form, or they can be administered separately in different dosage forms. When administered separately, administration may be by the same or a different route of administration for each of the APIs of the combination therapy.

Preferably, combination therapy provides a synergistic response. The term “synergistic” refers to the efficacy of the combination being more than the additive effects of either single therapy alone. The synergistic effect of combination therapy may permit the use of lower dosages and/or less frequent administration of at least one agent in the combination compared to its dose and/or frequency outside of the combination. The synergistic effect may also be manifested in the avoidance or reduction of adverse or unwanted side effects associated with the use of either therapy in the combination alone.

In embodiments, the administration of pharmaceutical composition as described here leads to the elimination of a symptom or complication of the disease or disorder being treated, however, elimination is not required. In one embodiment, the severity of the symptom is decreased. In the context of cancer, such symptoms may include clinical markers of severity or progression including the degree to which a tumor secrets growth factors, degrades the extracellular matrix, becomes vascularized, loses adhesion to juxtaposed tissues, or metastasizes, as well as the number of metastases.

### EXAMPLES

### Example 1

### Stability Studies

The reference solid oral dosage form of apilimod is a dry powder blend of excipients and apilimod dimesylate contained within a gelatin capsule. The active pharmaceutical ingredient, or API, apilimod dimesylate, is present at about 14 wt % in the powder blend with the remaining volume made up primarily by fillers such as microcrystalline cellulose (65%) and lactose (17%) with small amounts of additional excipients such as disintegrants, lubricants, and flow aids.

The dimesylate salt form of apilimod was initially selected for development due its high solubility in water (831 mg/mL) and physical stability. In particular, the dimesylate salt of apilimod was found to be stable for 4 years at controlled room temperature and for 6 months under accelerated conditions. For these tests, the apilimod dimesylate was stored in double polyethylene bags inside heat-sealed aluminum coated bags in a steel drum. Under ambient conditions of controlled temperature and relative humidity (RH) (25° C./60% RH), the percentage of drug substance remaining after 4 years was 98.7% and the amount of the 2-vinyl pyridine degradation product was less than 0.03% as assayed by high pressure liquid chromatography (HPLC). Even under “accelerated” conditions of 40° C./75% RH the apilimod dimesylate was stable out to 6 months, as evidenced by 99.7% drug substance remaining and an amount of 2-vinyl-pyridine less than 0.03%.

However, in contrast to the stability of the apilimod dimesylate salt form itself, the present inventors found that when blended with typical excipients such as fillers/bulking agents, disintegrants, lubricants, and flow processing aids, the apilimod began to degrade much more rapidly and generated unacceptable amounts of the degradation products 2-vinyl pyridine and STA-6066.

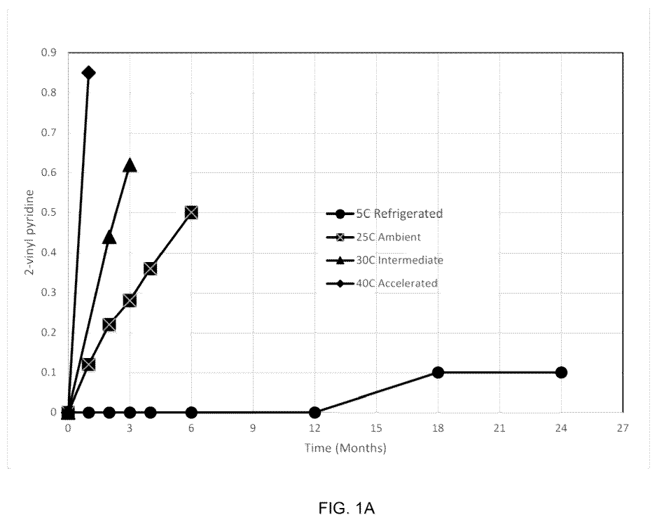

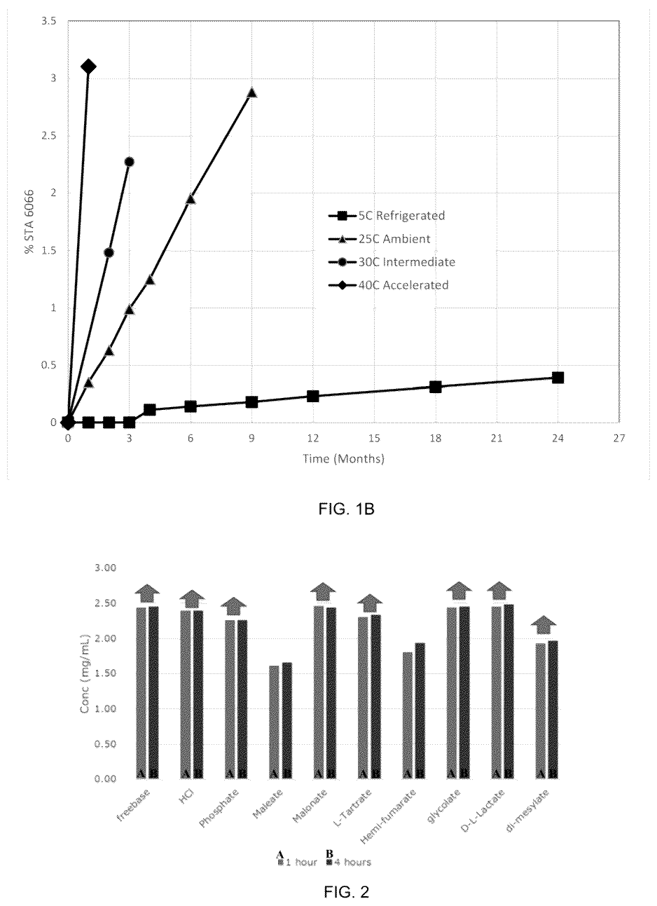

The rate of formation of 2-vinyl pyridine (FIG. 1A) and STA-6066 (FIG. 1B) was found to be temperature dependent. Under refrigerated conditions (5° C.) formation in the capsules was very slow, about 0.1% or less at 1 year and about 0.1-0.4% at 2 years, depending on the degradation product. However, storage of the capsules under ambient conditions (25° C./60% RH) resulted in a considerably faster rate of formation of both degradation products, 2-vinyl-pyridine and STA-6066. As shown in FIGS. 1A and 1B, initially the amount of each of 2-vinyl-pyridine and STA-6066 is less than 0.10% (below the limit of detection), but by 1 month the amount of both degradation products is detectable, with 2-vinyl-pyridine at about 0.12% and STA-6066 at about 0.35%. Both degradation products increase over time, to 0.71% for 2-vinylpyridine and 2.9% for STA-6066 by 9 months. Under accelerated storage conditions (40° C./75% RH) a similar but accelerated trend in the formation of 2-vinyl-pyridine and STA-6066 is observed. Thus, initially the amount of each of 2-vinyl-pyridine and STA-6066 is below the limit of detection (<0.10%) but the amount of each increases to 0.85% for 2-vinylpyridine and 3.1% for STA-6066 by 1 month. An intermediate trend in the formation of both degradation products was observed using intermediate conditions of 30° C./65% RH.

Thus, our stability studies indicated that the dimesylate form was unstable when formulated with common excipients in a powder blend, including under ambient conditions (25° C./60% RH) which are most desirable for long term storage. Although not wishing to be bound by any theory, we believe that this instability and the consequent formation of the undesirable degradation products may be caused by one or more aspects of the dimesylate salt form. First, this form is highly acidic, which might help catalyze the chemical fragmentation of apilimod to form the 2-vinyl-pyridine and STA-6066 compounds, as shown in the schematic below.

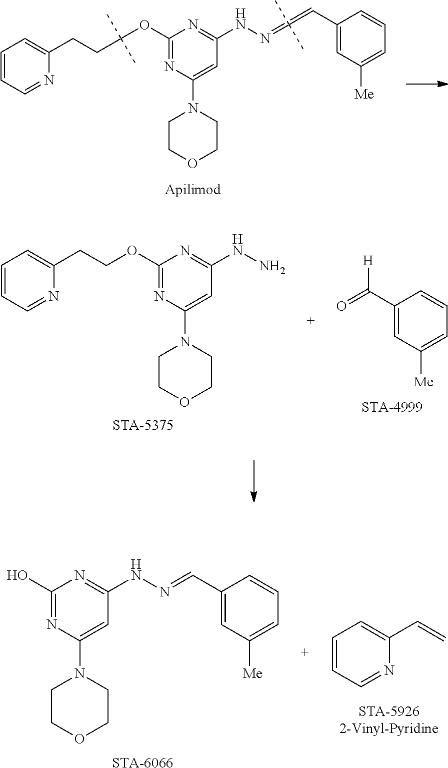

Second, the dimesylate salt form is so highly soluble that small but significant amounts of the apilimod dimesylate salt may dissolve in the trace amounts of water present in one or more of the excipients, providing a suitable aqueous environment for the chemical degradation of the apilimod to occur.

U.S. Pat. No. 7,745,436 teaches that a highly acidic salt such as the dimesylate is needed to provide the desired aqueous solubility of apilimod such that it is sufficiently bioavailable, for example when administered as an oral dosage form. The '436 patent teaches that acids with low pKa's (methanosulfonic acid (−1.2), HBr (−7), HCl (−4.5) and sulfuric (−3) are required for formation of a disalt with an excess (at least 2) equivalents of the strong acid. These disalts were found to have very high solubilities, for example the dimesylate salt had an aqueous solubility of 831 mg/mL and the dichloride 213 mg/mL.

These solubilities were ten times higher than the corresponding monosalts. In addition, the '426 patent teaches that the disalts are less prone to degradation (less colored) and have lower sensitivity to light (better light stability) compared to the monosalts. Accordingly, it was part of the common general knowledge as evidenced by the '436 patent, that the preferred pharmaceutically acceptable salts of apilimod were disalts formed from acids having a low pKa.

We first sought to identify other salts that would provide both a suitable crystalline form with apilimod and a 1:1 stoichiometry between the acid and the apilimod, while also being more stable than the dimesylate against degradation under ambient conditions (25° C./60% RH) when formulated with common excipients for a capsule dosage form. In addition, we sought to develop a formulation having an oral bioavailability similar to that of the highly soluble dimesylate salt form.

First, we identified acids having a pKa in the range of about 1-5 that were able to form suitable crystalline salts with apilimod and maintain a 1:1 stoichiometry between the acid and the apilimod. The acids shown in Table 1 below each formed a crystalline solid with a 1:1 ratio of acid to apilimod and each had a melting point above 130° C. In addition, these salts were physically stable against changes to the crystalline structure for at least 4 weeks under accelerated conditions of 50° C./75% RH, with no observable effects on crystallinity as measured by X-ray powder diffraction (XRPD) analysis or on melting point as measured by DSC. TGA analysis demonstrated that these salts were also non-solvated both at formation and for the duration of the 4 week stability testing.

TABLE 1
Acids Found to Afford Crystalline Solids with Apilimod

| | pKa of | MP of Salt with |
|---|---|---|
| Acid | Acid | Apilimod¹ |
| Hydrochloric | −6 | 191.9° C. |
| D,L-Lactate | 3.9 | 131.5° C. |
| Succinate | 4.2, 5.6 | 185.3° C. |
| Maleate | 1.9, 6.2 | 188.8° C. |
| Phosphate | 2.0, 7.1, 12.3 | 217.0° C. |
| Malonate | 2.8, 5.7 | 164.8° C. |
| L-Tartrate | 3.0, 4.4 | 190.5° C. |
| Fumarate | 3.0, 4.4 | 195.9° C. |
| 1-hydroxy-2- | 2.7, 13.5 | 179.4° C. |
| naphthoate | | |
| Glycolate | 3.3 | 134.8° C. |
| Bis-Mesylate | −1.9 | 206.3° C. |

¹As determined DSC analysis

### Methods

X-Ray Powder Diffraction (XRPD). XRPD diffractograms were acquired on PANalytical X'Pert Pro diffractometer using Ni-filtered Cu Ka (45 kV/40 mA) radiation and a step size of 0.03° 2theta and X'celerator™ RTMS (Real Time Multi-Strip) detector. Configuration on the incidental beam side: variable divergence slits (10 mm irradiated length), 0.04 rad Soller slits, fixed anti-scatter slit (0.50°), and 10 mm beam mask. Configuration on the diffracted beam side: variable anti-scatter slit (10 mm observed length) and 0.02 rad Soller slit. Samples were mounted flat on zero-background Si wafers.

Differential Scanning calorimetry (DSC). DSC was conducted with a TA Instruments Q100 or Q2000 differential scanning calorimeter equipped with an autosampler and a refrigerated cooling system under 40 mL/min N2 purge. DSC thermograms of screening samples were obtained at 15° C./min in crimped Al pans, unless noted otherwise. DSC thermograms of input and scaled-up materials were obtained at 10° C./min in crimped Al pans, unless noted otherwise.

Thermogravimetric Analysis (TGA). TGA thermograms were obtained with a TA Instruments Q50 thermogravimetric analyzer under 40 mL/min N2 purge in Pt or Al pans. TGA thermograms of screening samples were obtained at 15° C./min, unless noted otherwise. TGA thermograms of input and scaled-up material were obtained at 10° C./min, unless noted otherwise.

### Preparation of the HCl Salt

Acetone (50 mL; 20Vol) was added to the parent apilimod Lot 604004 (2.515 g; 6.010 mmol) and heated to 50° C., resulting into a solution. One equivalent of aqueous HCl (3M; 2.00 mL) was added followed by seed crystals of the HCl salt (Batch 103173-SU-01), resulting in rapid precipitation. The mixture was stirred at 50° C. for 2h and then stirred at RT overnight. Next day, a test aliquot was filtered and assayed by XRPD to confirm crystallinity. The remaining sample was filtered and washed with acetone and air-dried for 2h and then placed in oven at 50° C. under vacuum with nitrogen bleed for 2h. The experiment yielded 2.63 g (96.2%) of HCl salt.

### Preparation of the Phosphate Salt

Acetone (50 mL; 20Vol) was added to the parent apilimod Lot 604004 (2.514 g; 6.007 mmol) and heated to 50° C., resulting into a solution. One equivalent of aqueous phosphoric acid (3M; 2.00 mL) was added and a slurry was quickly observed followed by addition of seed crystals of the phosphate salt (Batch 103173-SU-10). The sample quickly precipitated. The mixture was stirred at 50° C. for 2h and then stirred at RT overnight. Next day, a test aliquot was filtered and assayed by XRPD to confirm crystallinity. The remaining sample was filtered and washed with acetone and air-dried for 2h and then placed in oven at 50° C. under vacuum with nitrogen bleed for 2h. The experiment yielded 2.46 g (79.2%) of phosphate salt.

### Preparation of the Maleate Salt

Acetone (60 mL; 20Vol) was added to the parent apilimod Lot 604004 (3.002 g; 7.173 mmol) and heated to 50° C., resulting into a solution. One equivalent of aqueous maleic acid (3M; 2.40 mL) was added, resulting in a thin slurry. Seed crystals of the maleate salt (Batch 103173-SU-04) were added. The sample was stirred at 50° C. for 2h followed by continued stirring for 3 more days at RT. Next day, a test aliquot was filtered and assayed by XRPD to confirm crystallinity. The remaining sample was filtered and washed with acetone and air-dried for 2h. The solids were observed to be slightly off white on the surface after filtration, while the remaining solids were white. The sample was placed in oven at 50° C. under vacuum with nitrogen bleed for 2h. The experiment yielded 2.91 g (75.9%) of maleate salt.

### Preparation of Malonate Salt

Acetone (50 mL; 20Vol) was added to the parent apilimod Lot 604004 (2.515 g; 6.011 mmol) and heated to 50° C., resulting into a solution. One equivalent of aqueous malonic acid (3M; 2.00 mL) was added, followed by seed crystals of malonate salt (Batch 103173-SU-11). A hazy solution was initially observed and slowly became a slurry. The mixture was stirred at 50° C. for 2 h followed by continued stirring at RT for three days. Next day, a test aliquot was filtered and assayed by XRPD to confirm crystallinity. The remaining sample was filtered and washed with acetone and air-dried for 2 h. Following day sample was placed in oven at 50° C. under vacuum with nitrogen bleed for 2 hrs. The experiment yielded 2.80 g (89.3%) of malonate salt.

### Preparation of L-Tartrate Salt

Acetone (51 mL; 20Vol) was added to the parent apilimod Lot 604004 (2.527 g; 6.038 mmol) and heated to 50° C., resulting in a solution. One equivalent of aqueous L-tartaric acid (3M; 2.013 mL) was added. A slurry was observed, and seed crystals of the L-tartrate salt (Batch 103173-SU-09) were added. The mixture was stirred at 50° C. for 2h followed by continued stirring at RT overnight. Next day, a test aliquot was filtered and assayed by XRPD to confirm crystallinity. The remaining sample was filtered and washed with acetone and air-dried for 2h and then placed in oven at 50° C. under vacuum with nitrogen bleed for 2h. The experiment yielded 3.26 g (94.8%) of L-tartrate salt.

### Preparation of Hemi-Fumarate Salt

Acetone (61 mL; 20Vol) was added to the parent apilimod Lot 604004 (3.067 g; 7.329 mmol) and heated to 50° C., resulting into a solution. One equivalent of fumaric acid (solid; 851 mg) was added along with seed crystals of the fumarate salt (Batch 103173-SU-06), and a faint yellow slurry was observed. The mixture was stirred at 50° C. for 2h followed by continued stirring at RT overnight. Next day, a test aliquot was filtered and assayed by XRPD to confirm crystallinity. The remaining sample was filtered and washed with acetone and air-dried for 2h and then placed in oven at 50° C. under vacuum with nitrogen bleed for 2h. The experiment yielded 3.45 g (98.8%) of hemi-fumarate salt.

### Preparation of DL-Lactate Salt

Acetonitrile (31 mL; 10Vol) was added to parent apilimod Lot 60404 (3.085 g; 7.370 mmol) and stirred at RT, resulting into a slurry. One equivalent of DL-Lactic acid neat (11.3M; 652.2 uL) was added followed by seed crystals of DL-lactate salt (Batch 103173-SU-13). The very thin slurry was concentrated to dryness in vacuo overnight. Acetonitrile (30 mL; 10Vol) was added to the solids followed by additional seed crystals of Batch 103173-SU-13. The slurry was stirred at RT for another day. Next day, a test aliquot was filtered and assayed by XRPD to confirm crystallinity. The remaining sample was filtered and washed with acetonitrile and air-dried for 2h and then placed in oven at 50° C. under vacuum with nitrogen bleed for 2 h. The experiment yielded 3.35 g (89.3%) of DL-Lactate salt.

### Preparation of DL-Lactate Salt

Acetonitrile with 2% water (30.6 mL total) was added to the DL-lactate salt Batch 103173-SU-21 (3.029 g; 5.96 mmol). The slurry was at RT for three days. A test aliquot was taken and assayed by XRPD to confirm crystallinity. The remaining sample was filtered and washed with acetonitrile and air-dried for 1.5 h and then placed in oven at 50° C. under vacuum with nitrogen bleed for 2 h. The experiment yielded 2.35 g (77.6%) of DL-lactate salt.

### Preparation of Glycolate Salt

Acetonitrile (51 mL; 10Vol) was added to parent apilimod Lot 604004 (5.081 g; 12.140 mmol) and stirred at RT, resulting into a slurry. One equivalent of glycolic acid (solid; 923 mg) was added followed by seed crystals of glycolate salt (Batch 103173-SU-14). The slurry stirred for two days at RT. A test aliquot was filtered and showed a significant amount of unreacted parent. The sample was then heated to 50° C. and stirred for 2h. An additional test aliquot was filtered and showed no improvement. An additional equivalent of glycolic acid as a solution was added (3M in THF; 4.05 mL), and the mixture was heated to 50° C. for 2h followed by continued stirring at RT overnight. The following day, a test aliquot was filtered and assayed by XRPD to confirm crystallinity. The remaining sample was filtered and washed with acetonitrile and air-dried for 2 h. Yielded weight 5.26 g (87.6%) of glycolate salt.

### Stability at Various pH

Next, the solubility of apilimod at various pH was evaluated. The samples were prepared at room temperature in 0.1N HCl (pH 1) and Britton-Robinson buffers (BRB) pH 2, 3, 4, 5, 6, 7 and 8. After 3 days, each sample was centrifuged for 30 min, the solution was filtered and the concentration of apilimod determined by HPLC analysis. HPLC analysis was carried out using an XTerra MS C18 (5 ?m 4.6×150 mm) column and 18-minute gradient HPLC conditions. The UV-spectrum of apilimod showed an absorbance maximum at 232, 260, and 332 nm. The results demonstrated that apilimod unexpectedly exhibits different solubility in aqueous media that is pH dependent. Thus, at pH of approximately 1 the solubility is more than 10 mg/mL, but the solubility decreases to 137 μg/mL at pH 2 and decreases still further at higher pH (Table 2).

TABLE 2
Solubility of Apilimod vs pH in 0.1N HCl and Britton Robinson Buffers

| | | | Day 3 | | |
|---|---|---|---|---|---|
| | Medium | Prep. | Conc | | |
| Medium | pH | (mg/mL) | (μg/mL) | pH | PXRD of Residuals |
| pH 1 | 1.08 | 11 | >10673 | 1.44 | In solution; no excess solid |
| (0.1N HCl) | | | | | |
| BRB pH 2 | 1.99 | 11 | 137 | 2.75 | Phosphate salt |
| BRB pH 3 | 2.88 | 2.8 | 17.3 | 3.61 | Phosphate salt |
| BRB pH 4 | 4.11 | 2.8 | 2.3 | 4.22 | Free base, hydrate Group B |
| BRB pH 5 | 5.04 | 2.8 | 0.2 | 5.08 | Free base, hydrate Group B |
| BRB pH 6 | 6.15 | 2.7 | <0.1 | 6.18 | Free base, hydrate Group B |
| BRB pH 7 | 7.18 | 2.7 | <0.1 | 7.21 | Free base, hydrate Group B |
| BRB pH 8 | 7.90 | 2.8 | <0.1 | 8.01 | Free base, hydrate Group B |

### Kinetic Solubility Studies

Kinetic solubility studies were carried out on the various apilimod salts provided in Table 1 in Fasted State Simulated Gastric Fluid (FaSSGF) at pH 1.6 at ambient temperature. FaSSGF is a dissolution medium that has the average acidic pH and similar osmolarity of fasted gastric fluid. FaSSGF that can help reveal how an oral drug is likely to behave in the stomach after drinking a glass of water. The stability, solubility and dissolution data generated from testing in FaSSGF can help identify critical factors influencing a drug's absorption in the fasted state, and in turn facilitate the selection of the appropriate solid state and formulation approach for a drug.

After 1 hour stirring at ambient temperature in FaSSGF at pH 1.6 at ambient temperature, 100% dissolution was observed in all samples, with the exception of maleate, and hemi-fumarate salts. The results are shown in FIG. 2. When tested in FaSSGF, residues for maleate, and hemi-fumarate salts were crystalline, and matched input salts by powder diffraction (PXRD) analysis.

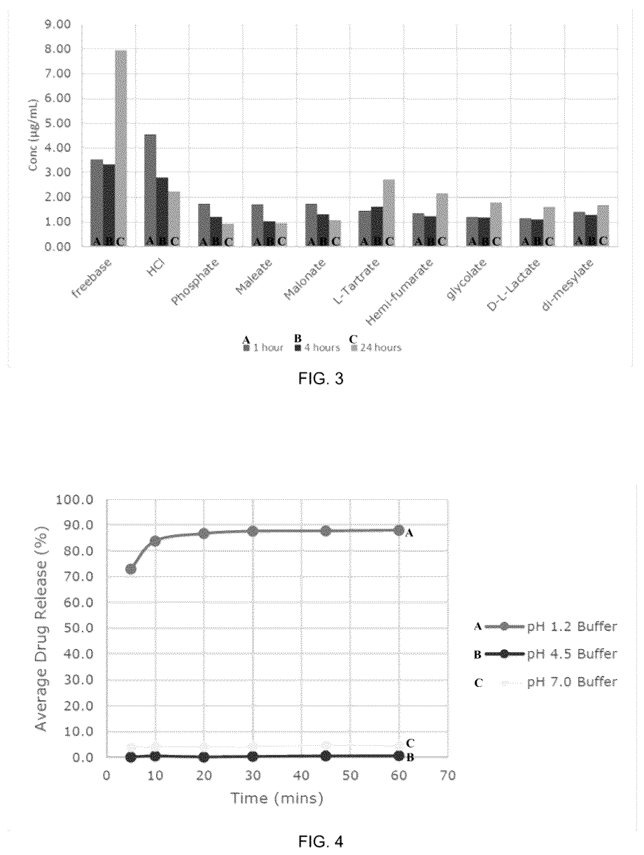

Additional kinetic solubility studies were carried out in Fasted State Simulated Intestinal Fluid (FaSSIF) at pH6.5 at ambient temperature and the results are shown in FIG. 3. FaSSIF helps reveal how an oral drug is likely to dissolve and potentially be absorbed in fluid from the upper intestine after drinking a glass of water. After 1 hour of stirring at ambient temperature, solubilities for all samples significantly decreased, resulting in μg/mL concentrations, which remained after 4 and 24 hours. After 1 hour of stirring, all samples appeared to be hazy suspensions, but after 4 and 24 hours their appearance changed to a milky white suspension by PXRD analysis. Other than the apilimod freebase, residues for all nine salts were crystalline, and did not match their respective input salts when tested in FaSSIF.

In summary, in FaSSGF, all salts were soluble at >2 mg/mL except for maleate and hemi-fumarate salts. In FaSSIF, solubilities of all salts were below 10 μg/mL, with the HCl salt exhibiting higher solubility at 1 hour compared to the other salts and the freebase.

### Intrinsic Dissolution Rates

Stationary disks for apilimod free base and the nine apilimod salts were prepared. Intrinsic dissolution rate of the free base and the nine salts were prepared from preparations at 3900 psi. Each disk was placed into a flat bottom vessel containing 700 mL of 0.01N HCl medium equipped with a Distek Dissolution bath, and a Distek cirvulator/heater set to 37° C. with a paddle speed of 50 rpm. The Opt-Diss 405 system single analytical wavelength settings was 333 nm with a background wavelength setting of 400 nm and reading were taken every 1 minute to 35 minutes. The representative intrinsic dissolution profiles for the salts and apilimod free base are provided in Table 3. Although the di-mesylate IDR profile is higher than the free base or the corresponding salts, the non-mesylate salts exhibit a dramatic difference in intrinsic dissolution rates, whereas the hemi-fumarate is less that 10-times lower than that D/L-Lactate and HCl salts. The kinetic solubility of the salt behavior is unpredictable and the dramatic difference between the non-mesylate salts was an unexpected discovery in the behavior of the salts.

TABLE 3
Intrinsic Dissolution Rate of Apilimod
Free Base and Apilimod Salts

| | Intrinsic Dissolution Rate |
|---|---|
| | (mg/(min*cm³) free base |
| Salt | equivalent |
| Free base | 0.3272 |
| HCl | 0.7924 |
| Phosphate | 0.1798 |
| Maleate | 0.0871 |
| Malonate | 0.2847 |
| L-Tartrate | 0.3399 |
| Hemi-fumarate | 0.0626 |
| Glycolate | 0.2670 |
| D/L-Lactate | 0.6366 |
| Di-Mesylate | 9.3410 |

At the end of the IDR analysis, the pellet surface was analyzed by PXRD and compared to its corresponding input API for any physical changes. The 8 non-mesylate salts and the free base residuals (Table 4) residuals were analyzed and the free base, HCl, phosphate, maleate, malonate, L-Tartrate, and hemi-fumarate and were found to match the XRPD pattern of the initial input material. The Glycolate residual XRPD pattern exhibited a pattern consistent with the input material and the poorly crystalline phase. The D/L-Lactate residuals XRPD pattern was consistent with the free base hydrate. The results indicate the HCl, which possesses the highest solubility also exhibits good physical stability during the dissolution process.

TABLE 4
PXRD Summary of Apilimod Free Base
and Salts after the IDR Experiment.

| | Residual Solids |
|---|---|
| Salt | By XRPD |
| Free base | Matches Input |
| HCl | Matches Input |
| Phosphate | Matches Input |
| Maleate | Matches Input |
| Malonate | Matches Input |
| L-Tartrate | Matches Input |
| Hemi-fumarate | Matches Input |
| Glycolate | Mixture of Input and poorly Crystalling |
| | Phase |
| D/L-Lactate | Free Base Hydrate |

In summary, these results demonstrate that the mono-salts of apilimod (HCl, phosphate, maleate, malonate, L-tartrate, and the hemi-fumarate) possess suitable solid state stability and intrinsic solubility for formulations of apilimod.

### Example 2

### Orally Disintegrating Tablets of the Apilimod Salts

In this study, micronized apilimod salts (hydrochloride, malonate, and L-tartrate salts) were incorporated into orally disintegrating tablets at 50 mg or 125 mg dose strengths. The following three salts were assessed at concentrations equivalent to 16.67% w/w for the apilimod free base: apilimod hydrochloride (d90=3.77 μm), apilimod malonate (d90=5.72 μm) and apilimod L-tartrate (d90=7.20 μm). The study was conducted to assess the wettability and dispersion behavior of the micronized salts during active pharmaceutical ingredient (API) addition, mixing and dosing and to measure the dissolution rate of the 125 mg dose strength product dosed at the 0-hour suspension hold (SH) timepoint. The dissolution testing were used to determine if the salt forms of the API can achieve 100% dissolution within the target time of 15 minutes. The formulation details are shown in Table 5 below.

TABLE 5
Example Formulation of Apilimod Salts

| | | | | |
|---|---|---|---|---|
| | | | | Weight |
| | | Theoretical | Theoretical | Dispensed for |
| | | mg/unit | mg/unit | 125 mg dose |
| Material | % w/w | (50 mg) | (125 mg) | strength (g) |

apilimod hydrochloride

| | | | | |
|---|---|---|---|---|
| micronized apilimod | 17.86 | 8.93 | 22.23 | 23.22 |
| hydrochloride | | | | |
| Gelatin | 3.7 | 1.85 | 4.63 | 4.81 |
| (Fish higher molecular | | | | |
| weight) | | | | |
| Mannitol | 2.8 | 1.4 | 3.5 | 3.64 |
| Purified Water* | 75.64 | 37.82 | 94.55 | 98.33 |
| Total | 100.00 | 50.00 | 125.01 | 130.00 |

apilimod malonate

| | | | | |
|---|---|---|---|---|
| micronized apilimod | 20.99 | 10.50 | 26,24 | 32.53 |
| malonate | | | | |
| Gelatin | 3.7 | 1.85 | 4.63 | 5.74 |
| (Fish higher molecular | | | | |
| weight) | | | | |
| Mannitol | 2.8 | 1.40 | 3.5 | 4.34 |
| Purified Water* | 72.51 | 36.26 | 4.34 | 112.39 |
| Total | 100.00 | 50.01 | 125.01 | 155.00 |

apilimod L-tartrate

| | | | | |
|---|---|---|---|---|
| micronized apilimod L- | 23.05 | 11.53 | 28.81 | 41.49 |
| tartrate | | | | |
| Gelatin | 3.7 | 1.85 | 4.63 | 6.66 |
| (Fish higher molecular | | | | |
| weight) | | | | |
| Mannitol | 2.8 | 1.40 | 3.50 | 5.04 |
| Purified Water* | 70.45 | 35.23 | 88.06 | 126.81 |
| Total | 100.00 | 50.01 | 125.00 | 180.00 |

apilimod free base

| | | | | |
|---|---|---|---|---|
| micronized apilimod | 20.83 | 10.42 | 26.04 | 47.91 |
| free base | | | | |
| Gelatin | 3.7 | 1.85 | 4.63 | 8.51 |
| (Fish higher molecular | | | | |
| weight) | | | | |
| Mannitol | 2.8 | 1.40 | 3.50 | 6.44 |
| sodium hydroxide (aq) | 5 (pH | 0.07 | 0.18 | 0.33 |
| | 7.0 ± 0.5) | | | |
| Purified Water* | 100.00% | 36.26 | 90.66 | 166.81 |
| Total | 100.00 | 50.00 | 125.01 | 230.00 |

*Purified water is removed during freeze drying (lyophilization)

### Synthesis of Orally Disintegrating Tablets

Orally disintegrating tablets of the tested apilimod salts were manufactured. The API incorporated easily into the tablets. Following mixing, the mixes appeared to have a smooth consistency, with no visible agglomerates or aeration. The mixes were dosed into blisters and frozen at −80.0° C. with a freeze tunnel residence time of 3 minutes and 15 seconds. The product was frozen after a single pass through the freeze tunnel. The frozen product was then transferred from the freeze tunnel to a freezer and stored below −15° C. until loading into the freeze dryer.

The particles were uniformly distributed across each batch and from samples dosed at both the 0-hour and 24-hour suspension hold (SH) timepoint. Apilimod malonate had a needle-like morphology whilst apilimod hydrochloride and apilimod L-tartrate particles were more irregularly shaped. No morphological change in the API was observed in the formulations over the 24-hour SH period.

### Dispersion Time

The dispersion time results of the orally disintegrating tablets of the tested apilimod salts are summarized in Table 6. The dispersion time is a measurement of the time required for a unit to fully wet when added to a beaker of water at approximately 20° C. As can be seen in the table, the dispersion times for all tested tablets are acceptable for an orally disintegrating tablet (ODT).

TABLE 6
Dispersion Time

| | | Range Dispersion | Mean Dispersion |
|---|---|---|---|
| | | Time | Time |
| API | Sub-Batch | (Seconds) (n = 5) | (Seconds) (n = 5) |
| Micronized apilimod | sub-batch 1 | <3 | <3 |
| hydrochloride | 50 mg dose strength | | |
| (17.86% w/w) | 0 SH | | |
| | sub-batch 2 | <5 | <5 |
| | 125 mg dose strength | | |
| | 0 SH | | |
| Micronized apilimod | sub-batch 1 | <3 | <3 |
| malonate | 50 mg dose strength | | |
| (20.99% w/w) | 0 SH | | |
| | sub-batch 2 | <3-<4 | <4 |
| | 125 mg dose strength | | |
| | 0 SH | | |
| Micronized apilimod | sub-batch 1 | <7-<12 | <10 |
| L-tartrate | 50 mg dose strength | | |
| (23.05% w/w) | 0 SH | | |
| | sub-batch 2 | <13-<15 | <15 |
| | 125 mg dose strength | | |
| | 0 SH | | |
| Micronized apilimod | sub-batch 1 | <3-<4 | <4 |
| hydrochloride | 50 mg dose strength | | |
| (17.86% w/w) | 24 SH | | |
| | sub-batch 2 | <4-<5 | <5 |
| | 125 mg dose strength | | |
| | 24 SH | | |
| Micronized apilimod | sub-batch 1 | N/A* | N/A* |
| malonate | 50 mg dose strength | | |
| (20.99% w/w) | 24 SH | | |
| | sub-batch 2 | <3 | <3 |
| | 125 mg dose strength | | |
| | 24 SH | | |
| Micronized apilimod | sub-batch 1 | <9-<11 | <11 |
| L-tartrate | 50 mg dose strength | | |
| (23.05% w/w) | 24 SH | | |
| | sub-batch 2 | <13-<16 | <9-<15 |
| | 125 mg dose strength | | |
| | 24 SH | | |
| Micronized apilimod | sub-batch 1 | <8-<9 | <9 |
| free base | 50 mg dose strength | | |
| (20.83% w/w; pH | 48 SH | | |
| 7.0 ± 0.5) | sub-batch 2 | <9-<10 | <10 |
| | 125 mg dose strength | | |
| | 48 SH | | |

*Batch not available

### Dissolution Testing

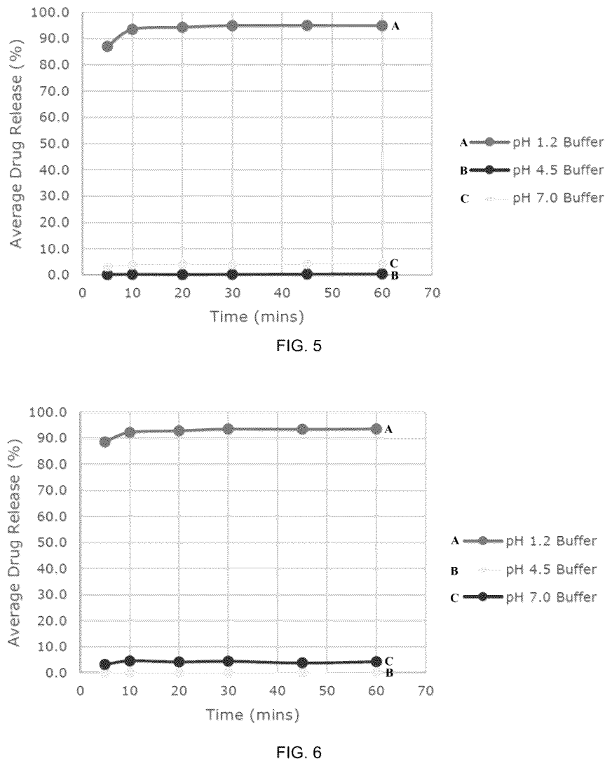

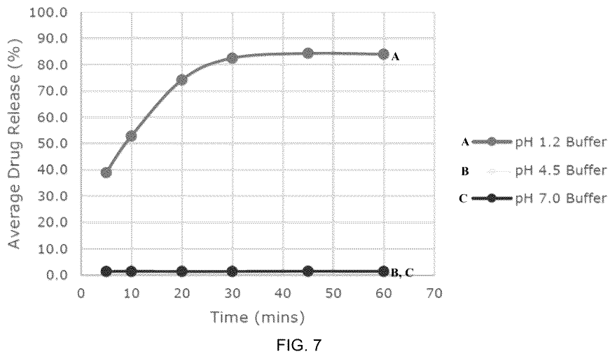

The results of the dissolution testing conducted on finished product samples are detailed in FIG. 4 (apilimod hydrochloride salt), FIG. 5 (apilimod malonate salt), and FIG. 6 (apilimod L-tartrate salt). The dissolution data for the free base units is further provided for comparison in FIG. 7. The dissolution tests for each salt were performed in hydrochloric acid buffer (pH 1.2), acetate buffer (pH 4.5) and phosphate buffer (pH 7.0). The dissolution of the finished product was assessed in 500 ml volumes of each buffer, and the percentage drug release was recorded after 5, 10, 20, 30, 45 and 60 minutes. Three replicates were conducted per dissolution medium. The results displayed in FIGS. 4-7 show the mean percentage drug release per timepoint for each buffer.

The results show that all batches had the best dissolution in pH 1.2 buffer. In contrast, limited dissolution was achieved in pH 4.5 and pH 7.0 buffer. For units containing either the apilimod malonate or L-tartrate salts, the mean percentage drug release was 93.3% and 92.0%, respectively at the 10-minute timepoint. Furthermore, the apilimod malonate and L-tartrate salts showed improved dissolution compared to the hydrochloride salt. For units containing the hydrochloride salt, the mean percentage drug release was 83.8% after 10 minutes. The dissolution also appeared to plateau at a later timepoint for the hydrochloride salt. However, the dissolution of all tested salts was superior to the free base (FIGS. 4-7).

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the disclosure, or aspects described herein, is/are referred to as comprising particular elements and/or features, certain embodiments described herein or aspects described herein consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments described herein, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment described herein can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A solid oral dosage form of apilimod comprising an apilimod salt and one or more pharmaceutically acceptable excipients, wherein the apilimod salt is a hydrochloride, malonate, or L-tartrate salt of apilimod and wherein the solid oral dosage form is fast-dissolving under acidic conditions.

2. The solid oral dosage form of apilimod of claim 1, wherein the apilimod salt is micronized.

3. The solid oral dosage form of apilimod of claim 1, wherein the solid oral dosage form further comprises gelatin and/or mannitol.

4. The solid oral dosage form of apilimod of claim 1, wherein the solid oral dosage form further comprises fish gelatin and mannitol.

5. The solid oral dosage form of apilimod of claim 1, wherein the solid oral dosage form comprises about 15-20% w/w of apilimod hydrochloride.

6. The solid oral dosage form of apilimod of claim 1, wherein the solid oral dosage form comprises about 18-22% w/w of apilimod malonate.

7. The solid oral dosage form of apilimod of claim 1, wherein the solid oral dosage form comprises about 21-25% w/w of apilimod tartrate.

8. The solid oral dosage form of claim 1, wherein the solid oral dosage form is a hard or soft gelatin capsule, a tablet, an oral dissolving tablet, oral disintegrating tablet (ODT) or a sublingual dosage form.

9. The solid oral dosage form of apilimod of claim 1, wherein the solid oral dosage form achieves at least 60%, at least 65%, at least 70%, at least 75%, at least 80%, at least 85%, at least 90%, at least 95%, or higher dissolution within 20 minutes under an acidic condition.

10. The solid oral dosage form of apilimod of claim 9, wherein the acidic condition has a pH of 1-2.

11. The solid oral dosage form of apilimod of claim 1, wherein the solid dosage form is stable for at least 1 month when stored under conditions of 25° C.

12. The solid oral dosage form of apilimod of claim 8, wherein the oral disintegrating tablet is at about 1-1,000 mg/unit dosage form.

13. The solid oral dosage form of apilimod of claim 8, wherein the oral disintegrating tablet is at about 25-500 mg/unit dosage form.

14. The solid oral dosage form of apilimod of claim 8, wherein the oral disintegrating tablet is at about 25 mg, 50 mg, 100 mg, 125 mg, 150 mg, 200 mg, 250 mg, 300 mg, 350 mg, 400 mg, 450 mg, or 500 mg/unit dosage form.

15. The solid oral dosage form of apilimod of claim 1, wherein the apilimod is selected from a formulation comprising:
i) about 18% w/w apilimod hydrochloride;
ii) about 21% w/w apilimod malonate;
iii) about 23% apilimod L-tartrate; or
iv) about 21% apilimod free base.

16. The solid oral dosage form of apilimod of claim 15, further comprising gelatin and mannitol.

17. Use of the solid oral dosage form of apilimod of claim 1 for the treatment of a neurodegenerative disease or disorder, a viral infection or cancer.

18. The use of claim 17, wherein the neurodegenerative disease or disorder is a dementia.

19. The use of claim 17, wherein the neurodegenerative disease or disorder is selected from Alzheimer's disease (AD), dementia pugilistica, diffuse Lewy body disease, frontotemporal dementia (FTD), amyotrophic lateral sclerosis (ALS), mixed dementia, senile dementia of Lewy body type, Parkinson's Disease, Huntingdon's disease, vascular dementia, frontotemporal dementia (FTD) or amyotrophic lateral sclerosis (ALS).

20. The use of claim 17, wherein the cancer is selected from brain cancer, breast cancer, cervical cancer, colorectal cancer, leukemia, lung cancer, lymphoma, non-Hodgkin's lymphoma, follicular lymphoma, melanoma or other skin cancer, ovarian cancer, prostate cancer, renal cancer, pancreatic cancer, liver cancer, or testicular cancer.

21. The solid oral dosage form of apilimod of claim 9, wherein the solid oral dosage form achieves at least 60%, at least 65%, at least 70%, at least 75%, at least 80%, at least 85%, at least 90%, at least 95%, or higher dissolution within about 20, 15, 10, or 5 minutes under an acidic condition.
